(19) United States
(12) Patent Application Publication
(10) Pub. No.: US 2004/0115647 A1
(43) Pub. Date: Jun. 17, 2004

(54) Apparatus and method for identifying biomarkers using a computer model

(76) Inventors: Thomas S. Paterson, West Hollywood, CA (US); Christina Maria Friedrich, San Francisco, CA (US); Leif Gustaf Wennerberg, Mountain View, CA (US); Seth Gary Michelson, San Jose, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/319,779

(22) Filed: Dec. 12, 2002

(51) Int. Cl. C12Q 1/68; G06F 19/00; G01N 33/48; G01N 33/50

(52) U.S. Cl. 435/6; 702/19

(57) ABSTRACT

An apparatus and method for identifying biomarkers using a computer model is described. In one embodiment, a computer-executable software code includes code to define a set of configurations associated with a computer model of a biological system. The computer-executable software code also includes code to apply a virtual measurement to the set of configurations to produce a result of the virtual measurement for each configuration of the set of configurations and code to apply a virtual therapy to the set of configurations to produce a result of the virtual therapy for each configuration of the set of configurations. The computer-executable software code further includes code to identify correlation between the results of the virtual measurement for the set of configurations and the results of the virtual therapy for the set of configurations.

### COPYRIGHT NOTICE

A portion of the disclosure of the patent document contains material that is subject to copyright protection. The copyright owner has no objection to the facsimile reproduction by anyone of the patent document, as it appears in the Patent and Trademark Office patent file or records, but otherwise reserves all copyright rights whatsoever.

### FIELD OF THE INVENTION

The present invention relates generally to computer models. More particularly, the present invention relates to identifying biomarkers using a computer model.

### BACKGROUND OF THE INVENTION

Biomarkers of therapies and of normal or disease conditions can be used for numerous applications in the life sciences field. For instance, a biomarker of a therapy typically refers to a biological attribute that can be associated with a particular effect of the therapy. More particularly, a biomarker of a therapy can refer to a biological attribute that can be evaluated to infer or predict a particular effect of the therapy. Biomarkers can be predictive of different effects of a therapy. For instance, biomarkers can be predictive of effectiveness, biological activity, safety, or side effects of a therapy.

Identification of biomarkers can play a key role in developing, testing, and implementing therapies to treat various diseases. For instance, a biomarker of a therapy can be evaluated for a human patient to predict the degree of effectiveness of the therapy for the human patient prior to a clinical trial. Such a biomarker can be used to select a group of human patients for the clinical trial, such that the clinical trial can target human patients that are likely to respond well to the therapy. Another biomarker of the therapy can be evaluated for a human patient during the course of the clinical trial to predict a surrogate end-point or outcome of the therapy for the human patient. Such a biomarker can be used to evaluate effectiveness of the therapy during the course of the clinical trial to determine, for example, whether to abort or alter the clinical trial. Yet another biomarker of the therapy can be evaluated for a human patient during the course of the clinical trial to assess biological activity of the therapy for the human patient.

In accordance with previous approaches, identification of biomarkers sometimes occurred during or after conclusion of a clinical trial based on statistical analysis of results of the clinical trial. However, such identification of biomarkers generally could not function to guide design of the clinical trial itself. In addition, without an identification of biomarkers prior to a clinical trial, appropriate measurements of the biomarkers may not be made during the course of the clinical trial, and potentially useful information regarding a therapy may not be obtained.

It is against this background that a need arose to develop the apparatus and method described herein.

### SUMMARY OF THE INVENTION

In one innovative aspect, the present invention relates to a computer-executable software code. In one embodiment, the computer-executable software code includes code to define a set of configurations associated with a computer model of a biological system. Each configuration of the set of configurations is associated with a different representation of the biological system. The computer-executable software code also includes code to apply a virtual measurement to the set of configurations to produce a result of the virtual measurement for each configuration of the set of configurations and code to apply a virtual therapy to the set of configurations to produce a result of the virtual therapy for each configuration of the set of configurations. The virtual measurement is associated with a measurement for the biological system absent a therapy, and the virtual therapy is associated with the therapy. The computer-executable software code further includes code to identify correlation between the results of the virtual measurement for the set of configurations and the results of the virtual therapy for the set of configurations.

### BRIEF DESCRIPTION OF THE DRAWINGS

For a better understanding of the nature and objects of the present invention, reference should be made to the following detailed description taken in conjunction with the accompanying drawings, in which:

FIG. 1 illustrates a system block diagram of a computer that can be operated in accordance with various embodiments of the invention.

FIG. 2 illustrates a flow chart for a process to identify one or more biomarkers of a therapy using a computer model, according to an embodiment of the invention.

FIG. 3 illustrates the architecture of a computer model that can be used to identify biomarkers in accordance with an embodiment of the invention.

FIG. 4 illustrates an example of a user-interface screen indicating a virtual patient that can be defined to represent a human patient.

FIG. 5 illustrates an example of a user-interface screen indicating another virtual patient that can be defined to represent a different human patient.

FIG. 6 illustrates an example of a user-interface screen indicating various stimulus-response tests that can be defined.

FIG. 7 illustrates an example of a user-interface screen indicating how a stimulus-response test can be defined.

FIG. 8 illustrates an example of a user-interface screen indicating various virtual measurements that can be defined.

FIG. 9 illustrates an example of a user-interface screen indicating plots of Forced Expiratory Volume in 1 second (FEV1) curves with respect to time.

FIG. 10 illustrates an example of a user-interface screen indicating how a virtual therapy can be defined.

FIG. 11 illustrates an example of a user-interface screen indicating various virtual measurements that can be defined to evaluate the behavior of a configuration of a computer model absent a virtual therapy and based on the virtual therapy.

FIG. 12 illustrates another example of a user-interface screen indicating various virtual measurements that can be defined to evaluate the behavior of a configuration of a computer model absent a virtual therapy and based on the virtual therapy.

FIG. 13 illustrates a flow chart to identify one or more biomarkers using a virtual therapy, according to an embodiment of the invention.

FIG. 14 illustrates an example of a user-interface screen that indicates results of virtual measurements for various virtual patients.

FIG. 15 illustrates an example of a graph that plots results of a first virtual measurement for multiple virtual patients with respect to results of a second virtual measurement for the multiple virtual patients.

### DETAILED DESCRIPTION OF THE INVENTION

### Overview

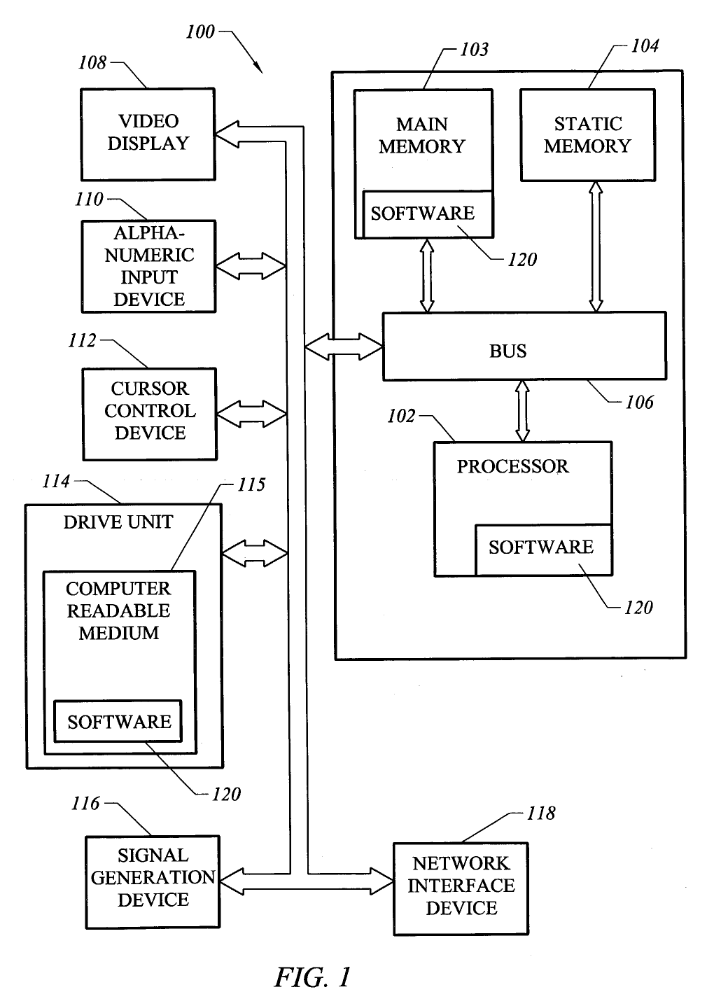

FIG. 1 illustrates a system block diagram of a computer 100 that can be operated in accordance with various embodiments of the invention. The computer 100 includes a processor 102, a main memory 103, and a static memory 104, which are coupled by bus 106. The computer 100 can also include a video display unit 108 (e.g., a liquid crystal display (LCD) or a cathode ray tube (CRT) display) on which a user-interface can be displayed. The computer 100 can further include an alpha-numeric input device 110 (e.g., a keyboard), a cursor control device 112 (e.g., a mouse), a disk drive unit 114, a signal generation device 116 (e.g., a speaker), and a network interface device 118. The disk drive unit 114 includes a computer-readable medium 115 storing software code 120 that implement processing according to some embodiments of the invention. The software code 120 can also reside within the main memory 103, the processor 102, or both. For certain applications, the software code 120 can be transmitted or received via the network interface device 118.

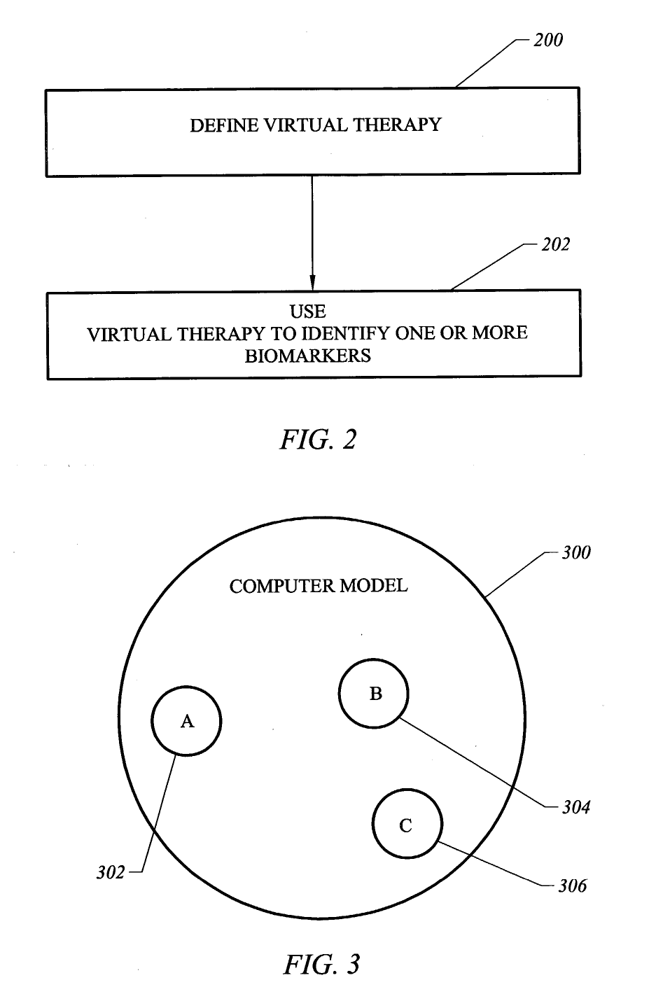

FIG. 2 illustrates a flow chart for a process to identify one or more biomarkers of a therapy using a computer model, according to an embodiment of the invention. In general, the computer model can represent any of a variety of systems that may be of interest to a user. Typically, the computer model will represent a system that is based on a real-world system. In the present embodiment of the invention, the computer model can represent a biological system to which the therapy can be applied. Examples of biological systems that can be represented by the computer model include a cell, a tissue, an organ, a multi-cellular organism, and a population of cellular or multi-cellular organisms.

The first step shown in FIG. 2 is to define a virtual therapy associated with the therapy (step 200). In the present embodiment of the invention, the virtual therapy can be defined to simulate the therapy. For certain applications, the virtual therapy can define a modification to the computer model to simulate the therapy.

The second step shown in FIG. 2 is to use the virtual therapy to identify one or more biomarkers of the therapy (step 202). In the present embodiment of the invention, a set (i.e., one or more) of virtual measurements can be defined. Each virtual measurement of the set of virtual measurements can be associated with a different measurement for the biological system. The set of virtual measurements can include virtual measurements that are configured to evaluate the behavior of the computer model absent the virtual therapy as well as based on the virtual therapy. In the present embodiment of the invention, the computer model can be executed to produce a set of results of the set of virtual measurements. Once produced, the set of results can be analyzed to identify one or more biomarkers of the therapy.

Computer Model

FIG. 3 illustrates the architecture of a computer model 300 that can be used to identify biomarkers of a therapy in accordance with an embodiment of the invention. As discussed previously, the computer model 300 can represent a biological system to which the therapy can be applied.

The computer model 300 can be defined as, for example, described in the patent to Paterson et al., entitled “Method of Managing Objects and Parameter Values Associated with the Objects Within a Simulation Model”, U.S. Pat. No. 6,078,739, issued on Jun. 20, 2000; the patent to Fink et al., entitled “Hierarchical Biological Modelling System and Method”, U.S. Pat. No. 5,657,255, issued on Aug. 12, 1997; the co-owned and co-pending patent application to Kelly et al., entitled “Method and Apparatus for Computer Modeling of an Adaptive Immune Response”, U.S. application Ser. No. 10/186,938, filed on Jun. 28, 2002; and the co-owned and co-pending patent application to Brazhnik et al., entitled “Method and Apparatus for Computer Modeling Diabetes”, U.S. application Ser. No. 10/040,373, filed on Jan. 9, 2002; the disclosures of which are incorporated herein by reference in their entirety. Alternatively, or in conjunction, the computer model 300 can be defined as in commercially available computer models such as, for example, Entelose Asthma PhysioLabg systems, Entelos® Obesity PhysioLab® systems, and Entelos® Adipocyte CytoLab™ systems.

In the present embodiment of the invention, the computer model 300 can include a mathematical model that represents a set of dynamic processes using a set of mathematical relations. In particular, the computer model 300 can represent a set of biological processes associated with the biological system using a set of mathematical relations. For instance, the computer model 300 can represent a first biological process using a first mathematical relation and a second biological process using a second mathematical relation. In at least one application, the computer model 300 can represent biological processes associated with an immune response to various antigens. A mathematical relation typically includes one or more variables the behavior (e.g., time evolution) of which can be simulated by the computer model 300. More particularly, mathematical relations of the computer model 300 can define interactions among variables, where the variables can represent biological attributes associated with inter-cellular constituents, cellular constituents, intra-cellular constituents, or a combination thereof, that make up the biological system. Constituents can include, for example, metabolites; DNA; RNA; proteins; enzymes; hormones; cells; organs; tissues; portions of cells, tissues, or organs; subcellular organelles; chemically reactive molecules like H⁺; superoxides; ATP; citric acid; protein albumin; as well as combinations or aggregate representations of these constituents. In addition, variables can represent various stimuli that can be applied to the biological system.

The behavior of variables can be influenced by a set of parameters included in the computer model 300. For example, parameters can include initial values of variables, half-lives of variables, rate constants, conversion ratios, exponents, and curve-fitting parameters. The set of parameters can be included in the mathematical relations of the computer model 300. In the present embodiment of the invention, parameters can be used to represent intrinsic properties (e.g., genetic factors or susceptibilities) as well as external influences (e.g., environmental factors) for the biological system.

The mathematical relations employed in the computer model 300 can include, for example, ordinary differential equations, partial differential equations, stochastic differential equations, differential algebraic equations, difference equations, cellular automata, coupled maps, equations of networks of Boolean, fuzzy logical networks, or a combination thereof. For certain applications, the mathematical relations used in the computer model 300 are ordinary differential equations that may take the form:

dx/dt=f(x, p, t),

where x is an N dimensional set of variables, t is time, dx/dt is the rate of change of x, p is an M dimensional set of parameters, and f is a function that represents interactions among the variables. In the present embodiment of the invention, the computer model 300 can be configured to simulate the behavior of variables by, for example, numerical or analytical integration of one or more mathematical relations. For example, numerical integration of the ordinary differential equations defined above can be performed to obtain values for the variables at various times.

For certain applications, the computer model 300 can be configured to allow visual representation of the mathematical relations as well as interrelationships between variables, parameters, and processes. This visual representation can include multiple modules or functional areas that, when grouped together, represent a large complex model of the biological system.

In the present embodiment of the invention, the computer model 300 can be used to define one or more configurations. With reference to FIG. 3, the computer model 300 is shown defining configuration A 302, configuration B 304, and configuration C 306. While three configurations are shown in FIG. 3, it should be recognized that more or less configurations can be defined depending on the specific application.

Various configurations of the computer model 300 can be associated with different representations of the biological system. In particular, various configurations of the computer model 300 can represent, for example, different variations of the biological system having different intrinsic properties, different external influences, or both. The observable condition (e.g., an outward manifestation) of the biological system can be referred to as its phenotype, while the underlying conditions of the biological system that give rise to the phenotype can be based on genetic factors, environmental factors, or both. As one of ordinary skill in the art will understand, phenotypes of a biological system can be defined with varying degrees of specificity. An example of such an observable condition or phenotype (e.g., a disease phenotype) might be an asthmatic condition or, more specifically, a moderate asthmatic condition that can be exhibited by an individual. A particular phenotype typically can be reproduced by different underlying conditions (e.g., different combinations of genetic and environmental factors). For example, while two individuals may appear to be similarly asthmatic, one could be asthmatic because of genetic factors, and the other could be asthmatic because of environmental factors. In the present embodiment of the invention, various configurations of the computer model 300 can be defined to represent different underlying conditions giving rise to a particular phenotype of the biological system. Alternatively, or in conjunction, various configurations of the computer model 300 can be defined to represent different phenotypes of the biological system.

For certain applications, various configurations of the computer model 300 may be referred to as virtual patients. For instance, configurations A 302, B 304, and C 306 may be referred to as virtual patients A, B, and C, respectively. A virtual patient can be defined to represent a human patient having a phenotype based on a particular combination of underlying conditions. Various virtual patients can be defined to represent human patients having the same phenotype but based on different underlying conditions. Alternatively, or in conjunction, various virtual patients can be defined to represent human patients having different phenotypes.

In the present embodiment of the invention, a configuration of the computer model 300 can be associated with a particular set of values for the parameters of the computer model 300. Thus, configuration A 302 may be associated with a first set of parameter values, and configuration B 304 may be associated with a second set of parameter values that differs in some fashion from the first set of parameter values. For instance, the second set of parameter values may include at least one parameter value differing from a corresponding parameter value included in the first set of parameter values. In a similar manner, configuration C 306 may be associated with a third set of parameter values that differs in some fashion from the first and second set of parameter values.

One or more configurations of the computer model 300 can be created based on an initial configuration that is associated with initial parameter values. A different configuration can be created based on the initial configuration by introducing a modification to the initial configuration. Such modification can include, for example, a parametric change (e.g., altering or specifying one or more initial parameter values), altering or specifying behavior of one or more variables, altering or specifying one or more functions representing interactions among variables, or a combination thereof. For instance, once the initial configuration is defined, other configurations may be created based on the initial configuration by starting with the initial parameter values and altering one or more of the initial parameter values. Alternative parameter values can be defined as, for example, disclosed in U.S. Pat. No. 6,078,739 discussed previously. These alternative parameter values can be grouped into different sets of parameter values that can be used to define different configurations of the computer model 300. For certain applications, the initial configuration itself can be created based on another configuration (e.g., a different initial configuration) in a manner as discussed above.

Alternatively, or in conjunction, one or more configurations of the computer model 300 can be created based on an initial configuration using linked simulation operations as, for example, disclosed in the co-pending and co-owned patent application to Paterson et al., entitled “Method and Apparatus for Conducting Linked Simulation Operations Utilizing A Computer-Based System Model”, U.S. application Ser. No. 09/814,536, filed Mar. 21, 2001, the disclosure of which is incorporated herein by reference in its entirety. This application discloses a method for performing additional simulation operations based on an initial simulation operation where, for example, a modification to the initial simulation operation at one or more times is introduced. In the present embodiment of the invention, such additional simulation operations can be used to create additional configurations of the computer model 300 based on an initial configuration that is created using the initial simulation operation. If desired, one or more simulation operations may be performed for a time sufficient to create one or more “stable” configurations of the computer model 300. Typically, a “stable” configuration is characterized by one or more variables under or substantially approaching equilibrium or steady-state condition.

For certain applications, various configurations of the computer model 300 can represent variations of the biological system that are sufficiently different to evaluate the effect of such variations on how the biological system responds to the therapy. In particular, one or more biological processes represented by the computer model 300 can be identified as playing a role in modulating biological response to the therapy, and various configurations can be defined to represent different modifications of the one or more biological processes. The identification of the one or more biological processes can be based on, for example, experimental or clinical data, scientific literature, results of a computer model, or a combination thereof. Once the one or more biological processes at issue have been identified, various configurations can be created by defining different modifications to one or more mathematical relations included in the computer model 300, which one or more mathematical relations represent the one or more biological processes. A modification to a mathematical relation can include, for example, a parametric change (e.g., altering or specifying one or more parameter values associated with the mathematical relation), altering or specifying behavior of one or more variables associated with the mathematical relation, altering or specifying one or more functions associated with the mathematical relation, or a combination thereof. The computer model 300 may be executed based on a particular modification for a time sufficient to create a “stable” configuration of the computer model 300.

A biological process that modulates biological response to the therapy can be associated with a knowledge gap or uncertainty, and various configurations of the computer model 300 can be defined to represent different plausible hypotheses or resolutions of the knowledge gap. By way of example, biological processes associated with airway smooth muscle (ASM) contraction can be identified as playing a role in modulating biological response to a therapy for asthma. While it may be understood that inflammatory mediators have an effect on ASM contraction, the relative effects of the different types of inflammatory mediators on ASM contraction as well as baseline concentrations of the different types of inflammatory mediators may not be well understood. For such a scenario, various configurations can be defined to represent human patients having different baseline concentrations of inflammatory mediators.

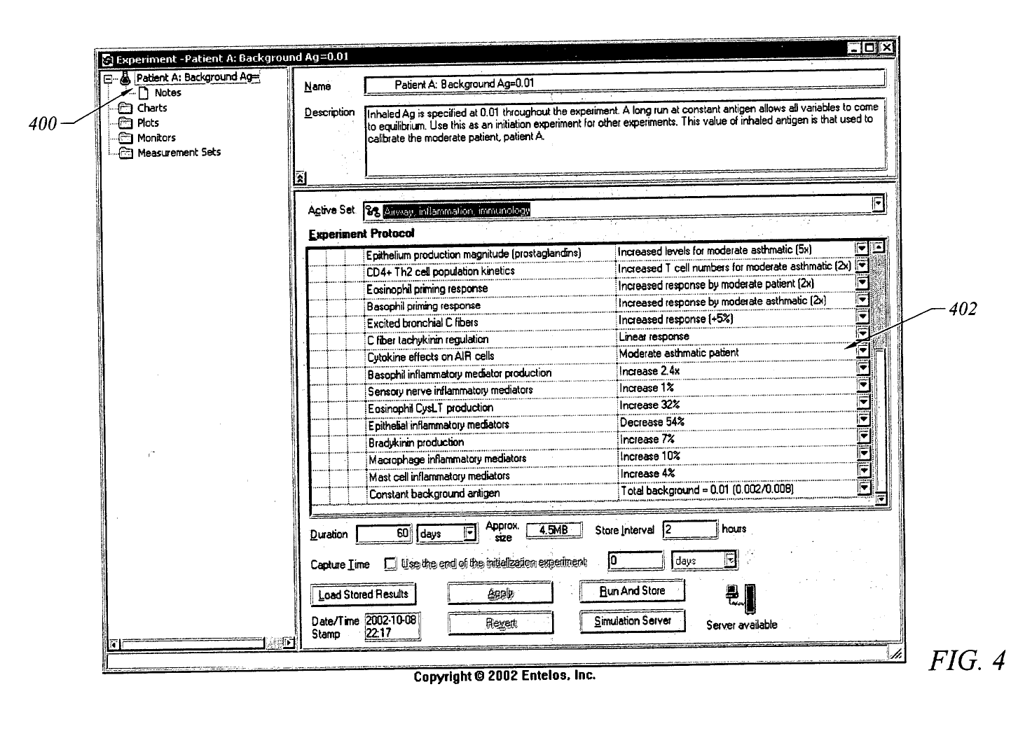

FIG. 4 illustrates an example of a user-interface screen indicating a virtual patient 400 that can be defined to represent a human patient. In this example, the virtual patient 400 labeled as “Patient A” is defined to represent a moderate asthmatic patient. As indicated in the “Experiment Protocol” window 402, various parameter values (e.g., parameter values associated with epithelium production magnitude, eosinophil priming response, constant background antigen (Ag), and so forth) can be specified for a simulation operation to create the virtual patient 400. In particular, parameters values associated with production levels for various types of inflammatory mediators can be specified to represent a moderate asthmatic patient having particular baseline concentrations of the various types of inflammatory mediators. As shown in the “Experiment Protocol” window 402, increased or decreased production levels can be specified for basophil inflammatory mediators, sensory nerve inflammatory mediators, eosinophil CysLT mediators, epithelial inflammatory mediators, bradykinin mediators, macrophage inflammatory mediators, and mast cell inflammatory mediators.

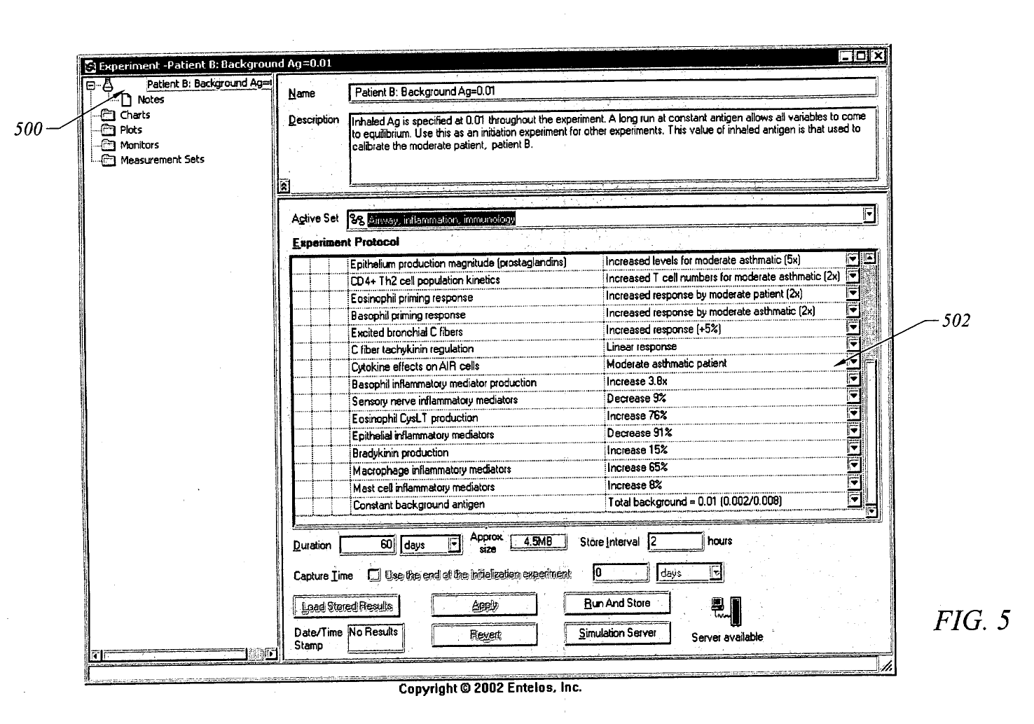

FIG. 5 illustrates an example of a user-interface screen indicating another virtual patient 500 that can be defined to represent a different human patient. In the present example, the virtual patient 500 labeled as “Patient B” is defined to represent a different moderate asthmatic patient. As indicated in the “Experiment Protocol” window 502, various parameter values (e.g., parameter values associated with epithelium production magnitude, eosinophil priming response, constant background antigen (Ag), and so forth) can be specified for a simulation operation to create the virtual patient 500. Here, a different set of parameter values associated with production levels for various types of inflammatory mediators is specified to represent a moderate asthmatic patient having different baseline concentrations of the various types of inflammatory mediators.

Referring back to FIG. 3, one or more configurations of the computer model 300 can be validated with respect to the biological system represented by the computer model 300. Validation typically refers to a process of establishing a certain level of confidence that the computer model 300 will behave as expected when compared to actual, predicted, or desired data for the biological system. For certain applications, various configurations of the computer model 300 can be validated with respect to one or more phenotypes of the biological system. For instance, configuration A 302 can be validated with respect to a first phenotype of the biological system, and configuration B 304 can be validated with respect to the first phenotype or a second phenotype of the biological system that differs in some fashion from the first phenotype.

One or more configurations of the computer model 300 can be validated using a set of virtual stimuli as, for example, disclosed in the co-pending and co-owned patent application to Paterson, entitled “Apparatus and Method for Validating a Computer Model”, U.S. application Ser. No. 10/151,581, filed May 16, 2002, the disclosure of which is incorporated herein by reference in its entirety. A virtual stimulus can be associated with a stimulus or perturbation that can be applied to a biological system. Different virtual stimuli can be associated with stimuli that differ in some fashion from one another. Stimuli that can be applied to a biological system can include, for example, existing or hypothesized therapeutic agents, treatment regimens, and medical tests. Additional examples of stimuli include exposure to existing or hypothesized disease precursors. Further examples of stimuli include environmental changes such as those relating to changes in level of exposure to an environmental agent (e.g., an antigen), changes in feeding behavior, and changes in level of physical activity or exercise.

For certain applications, a virtual stimulus may be referred to as a stimulus-response test. By applying a set of stimulus-response tests to a configuration of the computer model 300, a set of results of the set of stimulus-response tests can be produced. The configuration can be validated if the set of results of the set of stimulus-response tests sufficiently conforms to a set of expected results of the set of stimulus-response tests. An expected result of a stimulus-response test can be based on actual, predicted, or desired behavior of a biological system when subjected to a stimulus associated with the stimulus-response test. When validating one or more configurations of the computer model 300 with respect to a phenotype of the biological system, an expected result of a stimulus-response test typically will be based on actual, predicted, or desired behavior for the phenotype of the biological system. The behavior of a biological system can be, for example, an aggregate behavior of the biological system or behavior of a portion of the biological system when subjected to a particular stimulus. By way of example, an expected result of a stimulus-response test can be based on experimental or clinical behavior of a biological system when subjected to a stimulus associated with the stimulus-response test. For certain applications, an expected result of a stimulus-response test can include an expected range of behavior associated with a biological system when subjected to a particular stimulus. Such range of behavior can arise, for example, as a result of variations of the biological system having different intrinsic properties, different external influences, or both.

A stimulus-response test can be created by defining a modification to one or more mathematical relations included in the computer model 300, which one or more mathematical relations can represent one or more biological processes affected by a stimulus associated with the stimulus-response test. A stimulus-response test can define a modification that is to be introduced statically, dynamically, or a combination thereof, depending on the type of stimulus associated with the stimulus-response test. For example, a modification can be introduced statically by replacing one or more parameter values with one or more modified parameter values associated with a stimulus. Alternatively, or in conjunction, a modification can be introduced dynamically to simulate a stimulus that is applied in a time-varying manner (e.g., a stepwise manner or a periodic manner). For instance, a modification can be introduced dynamically by altering or specifying parameter values at certain times or for a certain time duration.

For certain applications, a stimulus-response test can be applied to one or more configurations of the computer model 300 using linked simulation operations as described in U.S. application Ser. No. 09/814,536 discussed previously. For instance, an initial simulation operation may be performed for a configuration, and, following introduction of a modification defined by a stimulus-response test, one or more additional simulation operations that are linked to the initial simulation operation may be performed for the configuration.

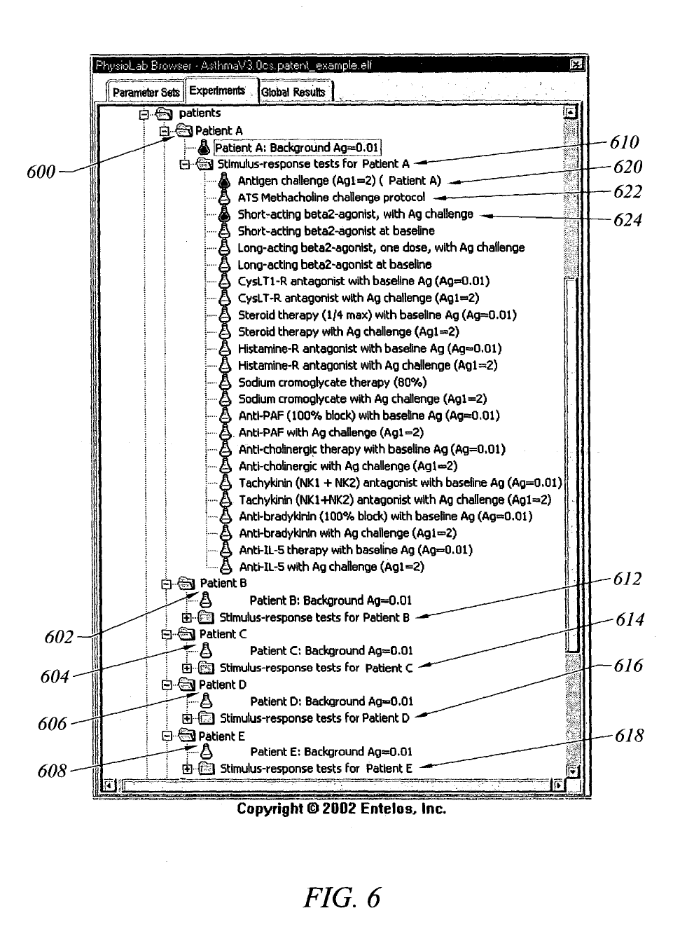

FIG. 6 illustrates an example of a user-interface screen indicating various stimulus-response tests that can be defined. As shown in FIG. 6, various stimulus-response tests (e.g., stimulus-response tests 620, 622, and 624) are grouped under a folder 610 labeled as “Stimulus-response tests for Patient A”. In the present example, the various stimulus-response tests can define modifications to a virtual patient 600 labeled as “Patient A” to simulate different stimuli that can be applied to a moderate asthmatic patient. In a similar manner, various stimulus-response tests can be grouped under folders 612, 614, 616, and 618 that are associated with virtual patients 602, 604, 606, and 608, respectively. In the present example, the folders 610, 612, 614, 616, and 618 can include one or more stimulus-response tests in common.

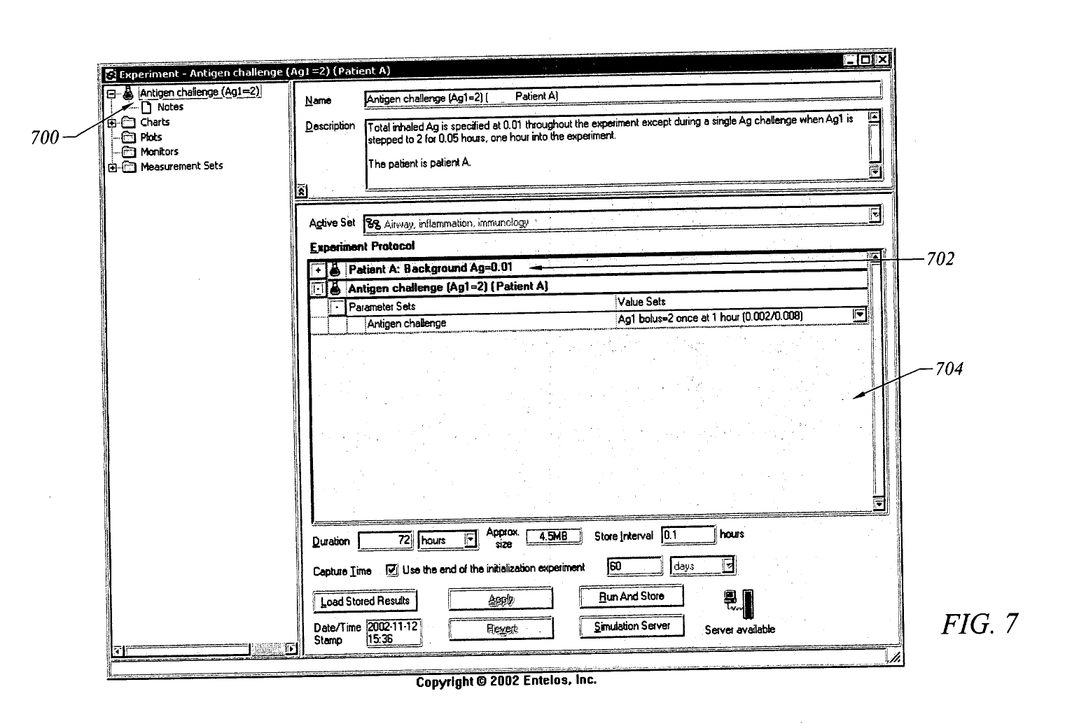

FIG. 7 illustrates an example of a user-interface screen indicating how a stimulus-response test 700 can be defined. As indicated in the “Experiment Protocol” window 704, the stimulus-response test 700 labeled as “Antigen challenge (Ag=2)” can define a modification to a virtual patient 702 labeled as “Patient A” to simulate an antigen challenge.

With reference to FIG. 3, a set of virtual measurements can be defined such that a set of results of a set of stimulus-response tests can be produced for a particular configuration of the computer model 300. Multiple virtual measurements can be defined, and a result can be produced for each of the virtual measurements. For certain applications, a virtual measurement can be associated with a measurement for a biological system. Examples of measurements can include existing or hypothesized measurements (e.g., experimental or clinical measurements) to evaluate various biological attributes of the biological system. Different virtual measurements can be associated with measurements that differ in some fashion from one another. For instance, different measurements can be configured to evaluate different biological attributes of a biological system. Alternatively, or in conjunction, different measurements can be configured to evaluate the same biological attribute of a biological system under different conditions (e.g., at different times).

In the present embodiment of the invention, a virtual measurement can be defined based on one or more variables of the computer model 300. As discussed previously, variables of the computer model 300 can represent various biological attributes of the biological system. For certain applications, a virtual measurement can simulate a measurement of a biological attribute and can be defined based on one or more variables that represent the biological attribute in the computer model 300. In the present embodiment of the invention, a virtual measurement can be defined based on the value of one or more variables or based on the value of a function of one or more variables. For example, virtual measurements can include a value at one or more times; an absolute or relative increase in a value over a time interval; an absolute or relative decrease in a value over a time interval; average value; minimum value; maximum value; time at minimum value; time at maximum value; area below a curve when values are plotted along a given axis (e.g., time); area above a curve when values are plotted along a given axis (e.g., time); pattern or trend associated with a curve when values are plotted along a given axis (e.g., time); rate of change of a value; average rate of change of a value; curvature associated with a value; number of instances a value exceeds, reaches, or falls below another value (e.g., a predefined value) over a time interval; minimum difference between a value and another value (e.g., a predefined value) over a time interval; maximum difference between a value and another value (e.g., a predefined value) over a time interval; a scaled value; a statistical measure associated with a value; as well as quantities based on combinations, aggregate representations, or relationships of two or more values (e.g., values of two or more different variables).

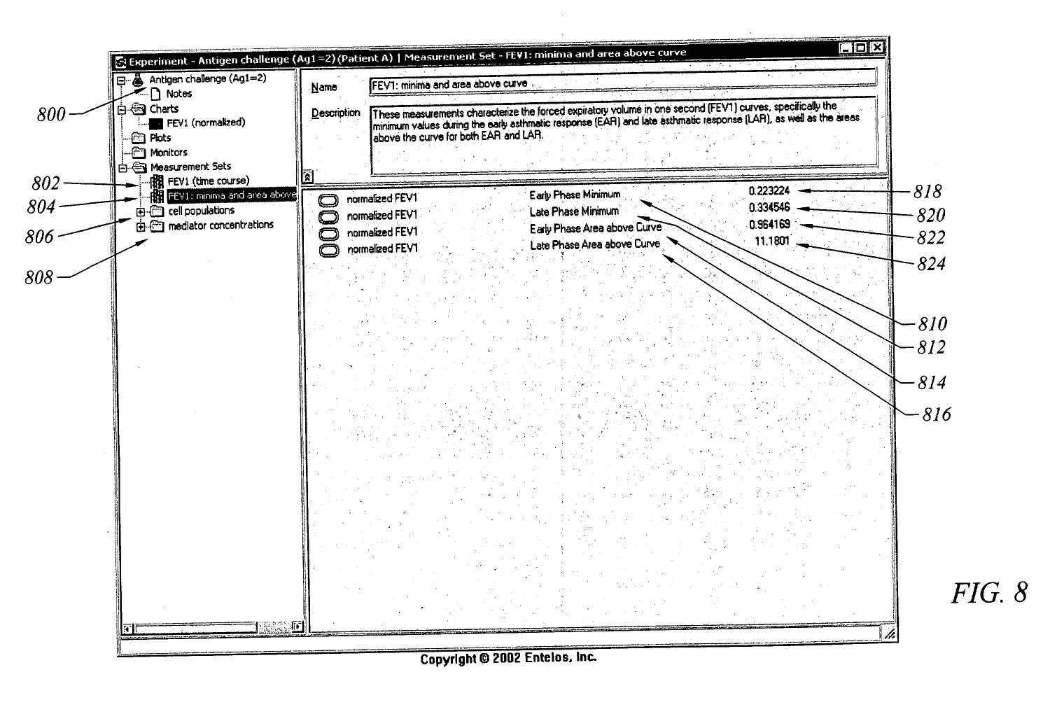

FIG. 8 illustrates an example of a user-interface screen indicating various virtual measurements that can be defined. In the present example, a stimulus-response test 800 labeled as “Antigen challenge (Ag1=2)” can be associated with a set of virtual measurements that are grouped under folders 802, 804, 806, and 808 labeled as “FEV1 (time course)”, “FEV1: minima and area above curve”, “cell populations”, and “mediator concentrations”, respectively. As shown in FIG. 8, virtual measurements 810, 812, 814, and 816 labeled as “Early Phase Minimum”, “Late Phase Minimum”, “Early Phase Area above Curve”, and “Late Phase Area above Curve” can be defined and are grouped under the folder 804. The virtual measurements 810, 812, 814, and 816 characterize the behavior of a Forced Expiratory Volume in 1 second (FEV 1) curve for a particular configuration of a computer model to which the stimulus-response test 800 is applied. In the present example, the virtual measurements 810, 812, 814, and 816 can be defined based on a variable that represents FEV1 in the computer model. As shown in FIG. 8, results 818, 820, 822, and 824 of the virtual measurements 810, 812, 814, and 816 can be produced based on applying the stimulus-response test 800 to the configuration. The results 818, 820, 822, and 824 of the virtual measurements 810, 812, 814, and 816 can be compared with expected results of the virtual measurements 810, 812, 814, and 816 to validate the configuration. In a similar manner, the stimulus-response test 800 can be applied to one or more additional configurations to validate the one or more additional configurations.

For certain applications, a configuration can be deemed to be validated with respect to a biological system if a certain number (e.g., a majority or all) of results of a set of results for the configuration are substantially consistent with expected results associated with the biological system. It should be recognized that a result of a stimulus-response test can be substantially consistent with an expected result without being identical to the expected result. For instance, a result of a stimulus-response test can be substantially consistent with an expected result if the difference between the two results falls within a certain range (e.g., within 20 percent or within 10 percent of the expected result). As another example, a result of a stimulus-response test can be substantially consistent with an expected result if the two results exhibit similar relative changes that can have different absolute values. As a further example, an expected result can include an expected range of behavior, and a result of a stimulus-response test can be substantially consistent with the expected result if the result of the stimulus-response test falls within the expected range of behavior.

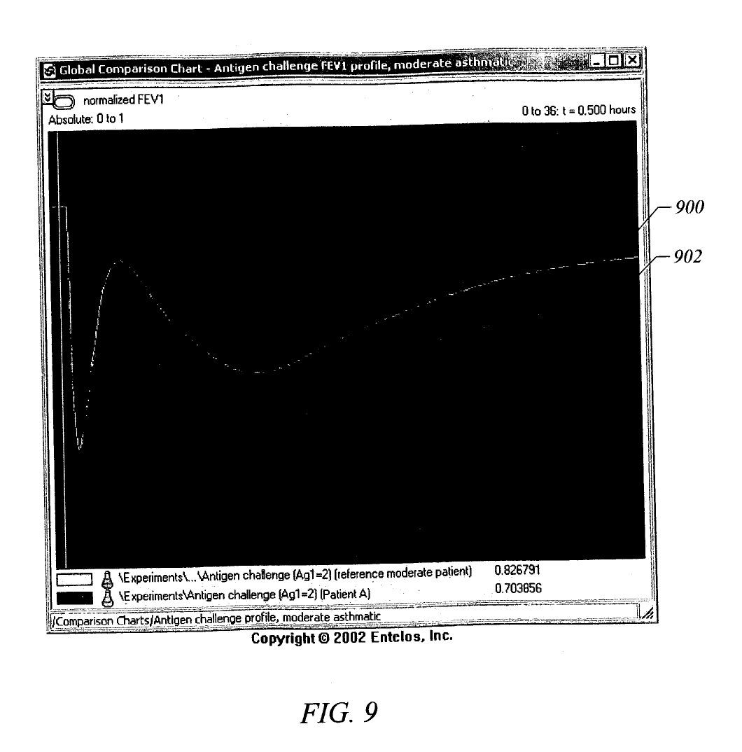

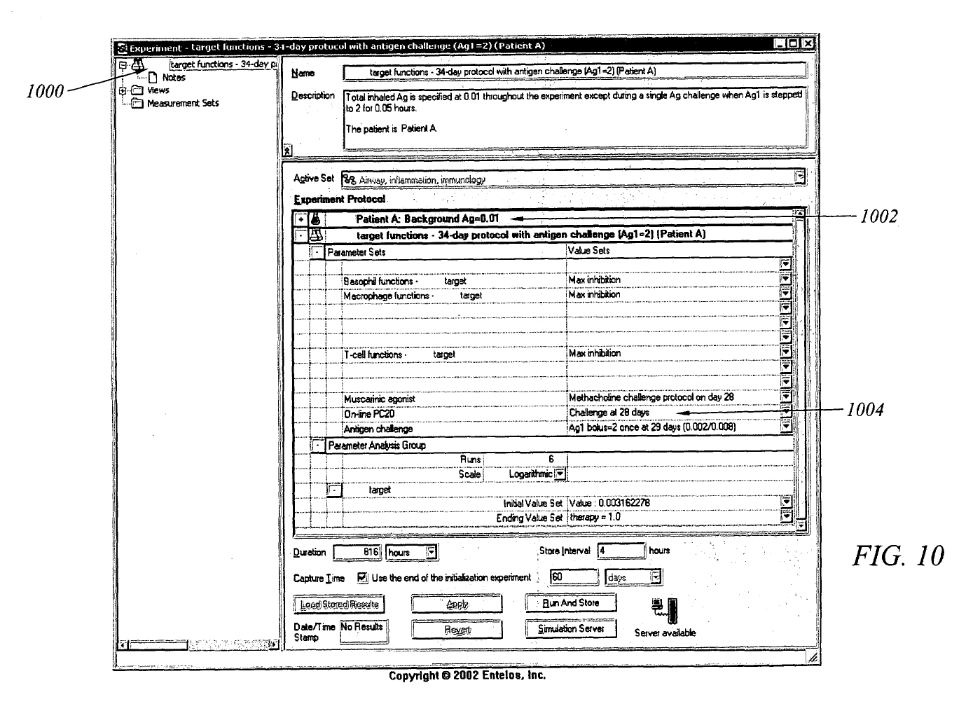

FIG. 9 illustrates an example of a user-interface screen indicating plots of FEV 1 curves 900 and 902 with respect to time. FEV1 curve 900 represents the behavior of a reference moderate asthmatic patient that is exposed to an antigen challenge. FEV1 curve 902 represents a result of a stimulus-response test simulating the antigen challenge for a particular configuration of a computer model. In the present example, FEV 1 curve 902 can be deemed to be substantially consistent with FEV1 curve 900, and the configuration can be validated with respect to the reference moderate asthmatic patient.

### Virtual Therapy

Once various configurations of a computer model are defined, the behavior of the various configurations can be used for predictive analysis. In particular, one or more configurations can be used to predict behavior of a biological system when subjected to various stimuli.

In the present embodiment of the invention, a virtual therapy associated with a therapy can be applied to a configuration in an attempt to predict how a real-world equivalent of the configuration would respond to the therapy. Therapies that can be applied to a biological system can include, for example, existing or hypothesized therapeutic agents and treatment regimens. By applying a virtual therapy to a configuration, a set of results of the virtual therapy can be produced, which set of results can be indicative of various effects of a therapy.

For certain applications, a virtual therapy can be created in a manner similar to that used to create a stimulus-response test. Thus, a virtual therapy can be created, for example, by defining a modification to one or more mathematical relations included in a computer model, which one or more mathematical relations can represent one or more biological processes affected by a therapy associated with the virtual therapy. A virtual therapy can define a modification that is to be introduced statically, dynamically, or a combination thereof, depending on the particular therapy associated with the virtual therapy. For certain applications, a virtual therapy can be applied to one or more configurations of a computer model using linked simulation operations as described in U.S. application Ser. No. 09/814,536 discussed previously.

FIG. 10 illustrates an example of a user-interface screen indicating how a virtual therapy 1000 can be defined. In the present example, the virtual therapy 1000 labeled as “target functions—34-day protocol with antigen challenge (Ag=2)” can define a modification to a virtual patient 1002 labeled as “Patient A” to simulate a therapy for asthma. As indicated in the “Experiment Protocol” window 1004, the virtual therapy 1000 can define a modification with respect to various parameter values (e.g., parameter values associated with basophil functions, macrophage functions, and T-cell functions) to simulate the therapy. In the present example, the virtual therapy 1000 can also define a modification to simulate application of muscarinic agonist, on-line PC20 (i.e., a methacholine challenge), and an antigen challenge to evaluate post-therapy behavior of the virtual patient 1002.

In the present embodiment of the invention, a set of virtual measurements can be defined such that a set of results of a virtual therapy can be produced for a particular configuration. Multiple virtual measurements can be defined, and a result can be produced for each of the virtual measurements. As discussed previously, a virtual measurement can be associated with a measurement for a biological system, and different virtual measurements can be associated with measurements that differ in some fashion from one another.

For certain applications, a set of virtual measurements can include a first set of virtual measurements and a second set of virtual measurements. The first set of virtual measurements can be defined to evaluate the behavior of one or more configurations absent the virtual therapy, while the second set of virtual measurements can be defined to evaluate the behavior of the one or more configurations based on the virtual therapy. The first and second set of virtual measurements can be associated with measurements configured to evaluate different biological attributes of a biological system. Alternatively, or in conjunction, the first and second set of virtual measurements can be associated with measurements configured to evaluate the same biological attributes of the biological system under different conditions. For instance, the first set of virtual measurements can include a first virtual measurement that is associated with a first measurement, and the second set of virtual measurements can include a second virtual measurement that is associated with a second measurement. In this example, the first measurement can be configured to evaluate a first biological attribute of the biological system absent the therapy, and the second measurement can be configured to evaluate the first biological attribute or a second biological attribute based on the therapy.

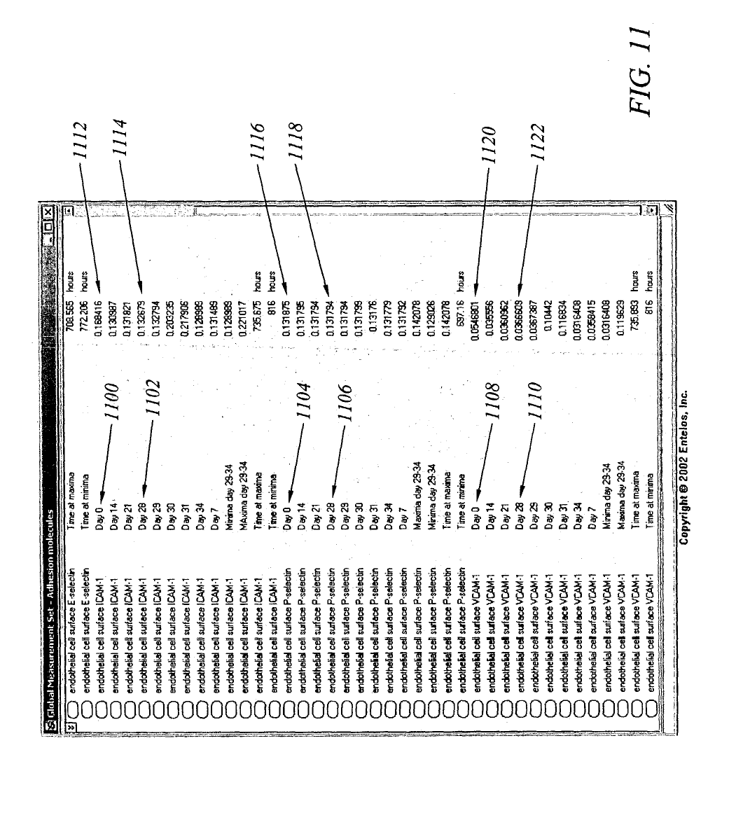

FIG. 11 illustrates an example of a user-interface screen indicating various virtual measurements (e.g., virtual measurements 1100, 1102, 1104, 1106, 1108, and 1110) that can be defined to evaluate the behavior of a configuration of a computer model absent a virtual therapy and based on the virtual therapy. In the present example, the various virtual measurements can be defined based on variables in the computer model that represent biological attributes associated with endothelial cell surface E-selectin, endothelial cell surface ICAM-1, endothelial cell surface P-selectin, and endothelial cell surface VCAM-1. As shown in FIG. 11, the various virtual measurements can be defined for various times. In the present example, the virtual measurements 1100, 1104, and 1108 are defined for an initial time (e.g., Day 0) and are configured to evaluate the behavior of the configuration absent the virtual therapy (e.g., prior to applying the virtual therapy at Day 0). Other virtual measurements (e.g., the virtual measurements 1102, 1106, and 1110) are defined for subsequent times (e.g., after Day 0) and are configured to evaluate the behavior of the configuration based on the virtual therapy. As shown in FIG. 11, various results (e.g., results 1112, 1114, 1116, 1118, 1120, and 1122) of the various virtual measurements can be produced for the configuration.

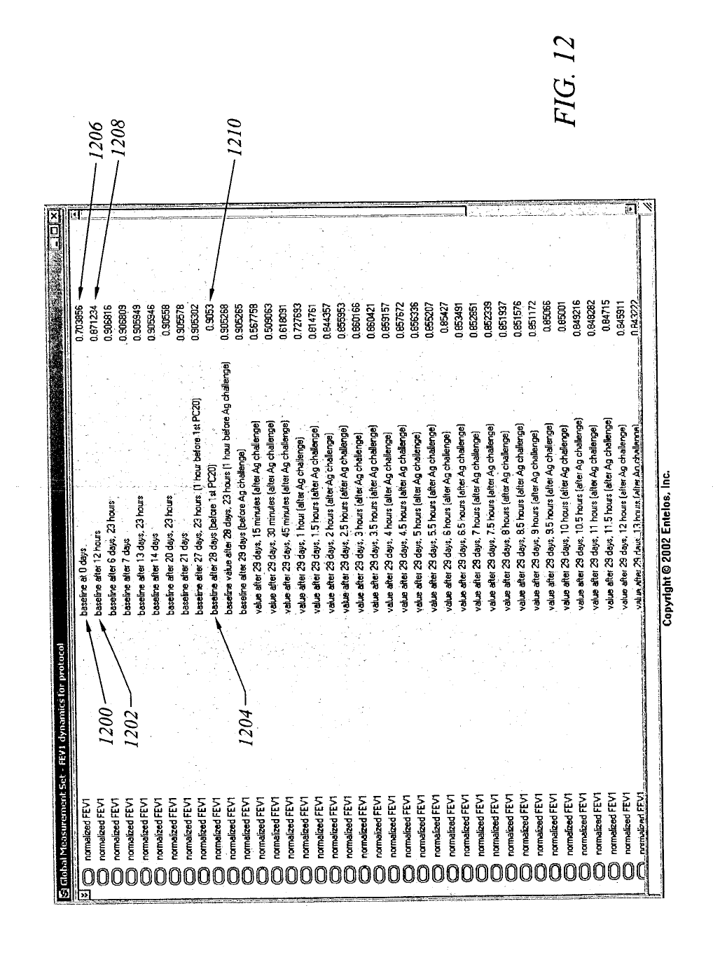

FIG. 12 illustrates another example of a user-interface screen indicating various virtual measurements (e.g., virtual measurements 1200, 1202, and 1204) that can be defined to evaluate the behavior of a configuration of a computer model absent a virtual therapy and based on the virtual therapy. In the present example, the various virtual measurements can be defined based on a variable in the computer model that represents FEV 1. Here, FEV 1 can be indicative of effectiveness of a therapy for asthma that is associated with the virtual therapy. As shown in FIG. 12, the virtual measurement 1200 is defined for an initial time (e.g., Day 0) and is configured to evaluate the behavior of the configuration absent the virtual therapy (e.g., prior to applying the virtual therapy at Day 0). Other virtual measurements (e.g., the virtual measurements 1202 and 1204) are defined for subsequent times (e.g., after Day 0) and are configured to evaluate the behavior of the configuration based on the virtual therapy. As shown in FIG. 12, various results (e.g., results 1206, 1208, and 1210) of the various virtual measurements can be produced for the configuration. For instance, result 1206 indicates a baseline FEV 1 value for the configuration absent the virtual therapy, while results 1208 and 1210 are indicative of effectiveness of the virtual therapy for the configuration after 12 hours and after 28 days, respectively.

### Using Virtual Therapy to Identify Biomarkers

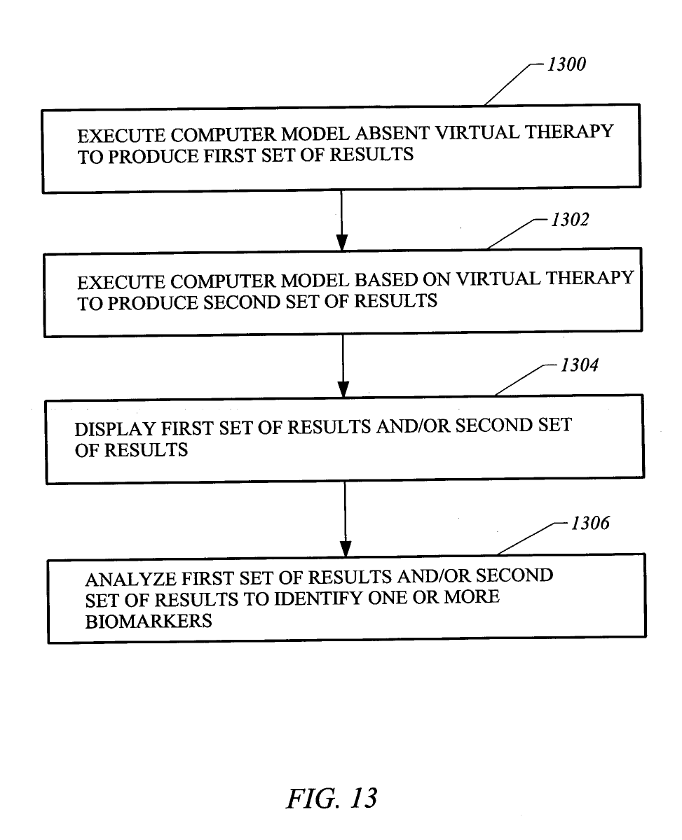

Once a virtual therapy is defined for a therapy, it can be used for the purpose of identifying one or more biomarkers of the therapy using a computer model. FIG. 13 illustrates a flow chart to identify one or more biomarkers using a virtual therapy, according to an embodiment of the invention.

The first step shown in FIG. 13 is to execute a computer model absent the virtual therapy to produce a first set of results (step 1300). In the present embodiment of the invention, a first set of virtual measurements can be defined to evaluate the behavior of one or more configurations of the computer model absent the virtual therapy. Accordingly, the first step (step 1300) can entail applying the first set of virtual measurements to one or more configurations to produce the first set of results. Each virtual measurement of the first set of virtual measurements can be associated with a different measurement for a biological system absent the therapy.

For certain applications, the first set of virtual measurements can be applied to multiple configurations of the computer model such that the first set of results can include results of the first set of virtual measurements for each configuration of the multiple configurations. The first set of virtual measurements may be applied to the multiple configurations simultaneously, sequentially, or a combination thereof. For instance, the first set of virtual measurements can be initially applied to a first configuration to produce results of the first set of virtual measurements for the first configuration. Subsequently, the first set of virtual measurements can be applied to a second configuration to produce results of the first set of virtual measurements for the second configuration. The first set of virtual measurements can be sequentially applied to the multiple configurations in accordance with an order that may be established by default or selected in accordance with a user-specified selection.

In the present embodiment of the invention, various mathematical relations of the computer model can be solved numerically by a computer using standard algorithms as, for example, disclosed in William H. Press et al. Numerical Recipes in C: The Art of Scientific Computing, 2nd edition (January 1993) Cambridge Univ. Press. For example, numerical integration of the ordinary differential equations defined previously can be performed to produce values of variables at one or more times. Such values of the variables can, in turn, be used to produce the first set of results of the first set of virtual measurements.

For certain applications, one or more results of the first set of results can be produced based on one or more virtual stimuli. For instance, the first step (step 1300) can entail applying a virtual stimulus to one or more configurations of the computer model to produce the first set of results. The virtual stimulus can be associated with a stimulus that differs in some fashion from the therapy. In the present embodiment of the invention, various mathematical relations of the computer model, along with a modification defined by the virtual stimulus, can be solved numerically by a computer using standard algorithms to produce values of variables at one or more times based on the modification. Such values of the variables can, in turn, be used to produce the first set of results of the first set of virtual measurements.

With reference to FIG. 13, the second step shown is to execute the computer model based on the virtual therapy to produce a second set of results (step 1302). In the present embodiment of the invention, a second set of virtual measurements can be defined to evaluate the behavior of one or more configurations of the computer model based on the virtual therapy. Accordingly, the second step (step 1302) can entail applying the second set of virtual measurements to one or more configurations to produce the second set of results. Each virtual measurement of the second set of virtual measurements can be associated with a different measurement for a biological system based on the therapy. In the present embodiment of the invention, the first and second set of virtual measurements can be associated with measurements configured to evaluate different biological attributes of a biological system. Alternatively, or in conjunction, the first and second set of virtual measurements can be associated with measurements configured to evaluate the same biological attributes of the biological system under different conditions.

For certain applications, the virtual therapy can be applied to multiple configurations of the computer model such that the second set of results can include results of the second set of virtual measurements for each configuration of the multiple configurations. The virtual therapy may be applied to the multiple configurations simultaneously, sequentially, or a combination thereof. For instance, the virtual therapy can be sequentially applied to the multiple configurations in accordance with an order that may be established by default or selected in accordance with a user-specified selection.

In the present embodiment of the invention, various mathematical relations of the computer model, along with a modification defined by the virtual therapy, can be solved numerically by a computer using standard algorithms to obtain values of variables at one or more times based on the modification. Such values of the variables can, in turn, be used to produce the second set of results of the second set of virtual measurements.

With reference to FIG. 13, the third step shown is to display one or both of the first set of results and the second set of results (step 1304). In the present embodiment of the invention, a result can be displayed for each virtual measurement of the first and second set of virtual measurements. By displaying results for one or more configurations, the behavior of the one or more configurations can be evaluated to identify one or more biomarkers. For certain applications, reports, tables, or graphs can be provided to facilitate understanding by a user.

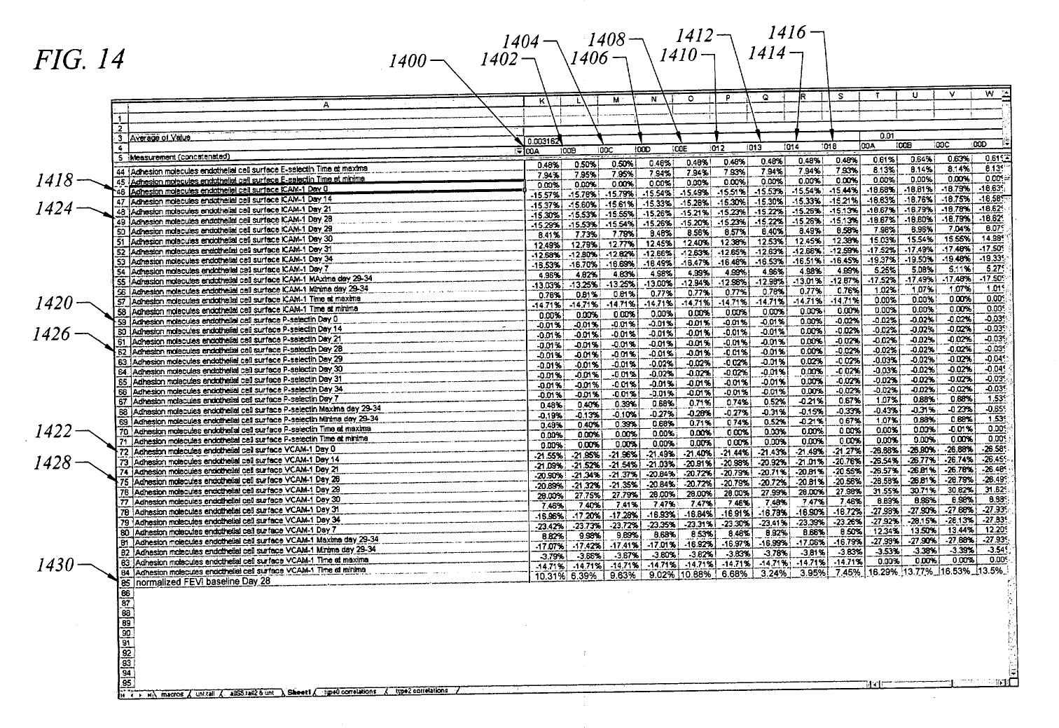

Results for multiple configurations can be displayed to allow comparative analysis across different configurations. FIG. 14 illustrates an example of a user-interface screen that indicates results of virtual measurements for various virtual patients. In the present example, results are shown for nine virtual patients 1400, 1402, 1404, 1406, 1408, 1410, 1412, 1414, and 1416. Here, the virtual patients 1400-1416 can be defined to represent different moderate asthmatic patients. As shown in FIG. 14, various virtual measurements (e.g., virtual measurements 1418, 1420, 1422, 1424, 1426, 1428, and 1430) can be defined to evaluate the behavior of the virtual patients 1400-1416 absent a virtual therapy as well as based on the virtual therapy, and results of the various virtual measurements are shown for each virtual patient. In the present example, results of the various virtual measurements are expressed as a percentage change relative to an initial value (e.g., value at Day 0). The virtual measurement 1430 labeled as “normalized FEV1 baseline Day 28” can be defined based on a variable in the computer model that represents FEV 1 and can be configured to evaluate effectiveness of the virtual therapy for each virtual patient at Day 28. As shown in FIG. 14, results of the virtual measurement 1430 differ across the virtual patients 1400-1416.

Turning back to FIG. 13, the fourth step shown is to analyze one or both of the first set of results and the second set of results to identify one or more biomarkers (step 1306). For certain applications, identification of a biomarker can be made by a user evaluating the various results. Alternatively, or in conjunction, identification of a biomarker can be made automatically, and an indication can be provided to indicate whether the biomarker is identified.

In the present embodiment of the invention, the analysis implemented for the fourth step (step 1306) can depend on the particular biomarker to be identified. For certain biomarkers, the fourth step (step 1306) can entail comparing the first set of results with the second set of results. More particularly, the fourth step (step 1306) can entail comparing results of the first set of virtual measurements for one or more configurations with results of the second set of virtual measurements for the one or more configurations. For instance, the first set of virtual measurements can include a first virtual measurement, and the second set of virtual measurements can include a second virtual measurement. The first virtual measurement can be associated with a first measurement configured to evaluate a first biological attribute of a biological system absent the therapy, and the second virtual measurement can be associated with a second measurement configured to evaluate a second biological attribute of the biological system based on the therapy. Here, the second biological attribute can be indicative of a particular effect of the therapy (e.g., effectiveness, biological activity, safety, or side effect of the therapy). Results of the first virtual measurement for multiple configurations can be compared with results of the second virtual measurement for the multiple configurations. More particularly, comparing the results of the first virtual measurement for the multiple configurations with the results of the second virtual measurement for the multiple configurations can entail determining whether the results of the first virtual measurement are correlated with the results of the second virtual measurement. The first biological attribute can be identified as a biomarker that is predictive of the particular effect of the therapy based on determining that the results of the first virtual measurement are substantially correlated with the results of the second virtual measurement.

While a specific example of analyzing results of two virtual measurements (e.g., the first and second virtual measurements) is provided above, it should be recognized that, in general, results of two or more virtual measurements can be analyzed to identify a biomarker. For instance, the first set of virtual measurements can also include a third virtual measurement that is associated with a third measurement for the biological system, and the third measurement can be configured to evaluate a third biological attribute of the biological system absent the therapy. In the present example, results of the first and third virtual measurements for multiple configurations can be compared with results of the second virtual measurement for the multiple configurations. A combination of the results of the first and third virtual measurements can be determined to be substantially correlated with the results of the second virtual measurement, and a combination of the first and third biological attributes can be identified as a “multi-factorial” biomarker that is predictive of the particular effect of the therapy.

In the present embodiment of the invention, results of two or more virtual measurements can be determined to be substantially correlated based on one or more standard statistical tests. Statistical tests that can be used to identify correlation can include, for example, linear regression analysis, nonlinear regression analysis, and rank correlation test. In accordance with a particular statistical test, a correlation coefficient can be determined, and correlation can be identified based on determining that the correlation coefficient falls within a particular range. Examples of correlation coefficients include goodness of fit statistical quantity r² associated with linear regression analysis and Spearman Rank Correlation coefficient rₛ associated with rank correlation test.

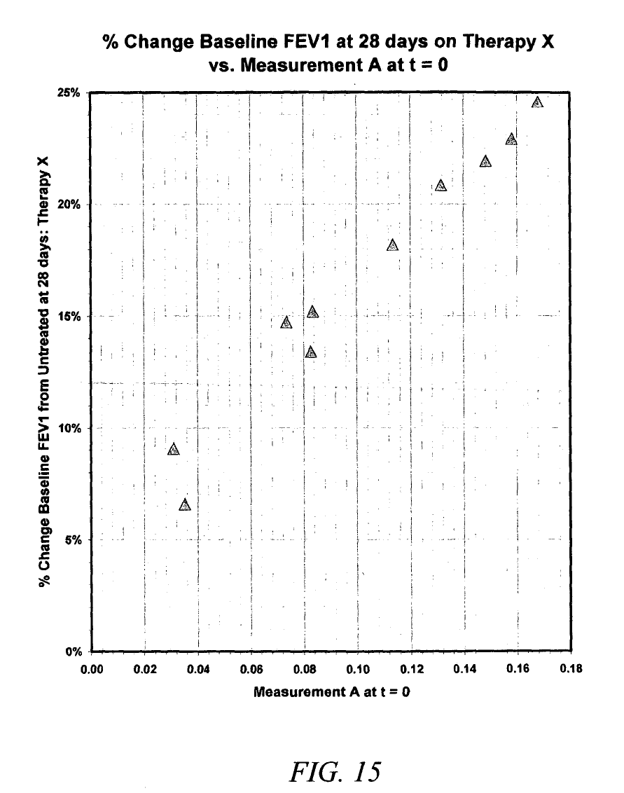

FIG. 15 provides an example of a graph that plots results of a first virtual measurement for various virtual patients with respect to results of a second virtual measurement for the various virtual patients. In the present example, the first virtual measurement labeled as “Measurement A at t=0” is associated with a first measurement that is configured to evaluate a first biological attribute of asthmatic patients absent a therapy for asthma. The second virtual measurement labeled as “% Change Baseline FEV1 from Untreated at 28 days: Therapy X” is associated with a second measurement that is configured to evaluate a second biological attribute of asthmatic patients subjected to the therapy. Here, the second biological attribute is associated with FEV1 and is indicative of effectiveness of the therapy. In the present example, the results of the first virtual measurement can be determined to be substantially correlated with the results of the second virtual measurement based on a correlation coefficient. Accordingly, the first biological attribute can be identified as a biomarker that is predictive of effectiveness of the therapy for asthmatic patients. In particular, the first biological attribute can be evaluated for a particular asthmatic patient prior to applying the therapy to predict the degree of effectiveness of the therapy for the particular asthmatic patient. In the present example, the graph predicts that a greater degree of effectiveness of the therapy can be achieved for the particular asthmatic patient if a greater measured value for the first biological attribute is obtained.

For other biomarkers, the fourth step (step 1306) shown in FIG. 13 can entail comparing results of two or more virtual measurements of the second set of virtual measurements. For instance, the second set of virtual measurements can include a first virtual measurement and a second virtual measurement. The first virtual measurement can be associated with a first measurement configured to evaluate a first biological attribute of a biological system based on the therapy, and the second virtual measurement can be associated with a second measurement configured to evaluate a second biological attribute of the biological system based on the therapy. Here, the second biological attribute can be indicative of a particular effect of the therapy. In the present example, results of the first virtual measurement for one or more configurations can be compared with results of the second virtual measurement for the one or more configurations. In particular, results of the first virtual measurement for multiple configurations can be compared with results of the second virtual measurement for the multiple configurations. Comparing the results of the first virtual measurement for the multiple configurations with the results of the second virtual measurement for the multiple configurations can entail determining whether the results of the first virtual measurement are correlated with the results of the second virtual measurement. The first biological attribute can be identified as a biomarker that is predictive of the particular effect of the therapy based on determining that the results of the first virtual measurement are substantially correlated with the results of the second virtual measurement. For certain applications, the first biological attribute can be identified as a biomarker that is predictive of effectiveness of the therapy and can be evaluated for the biological system during the course of the therapy to infer a surrogate end-point or outcome of the therapy.

For still other biomarkers, the fourth step (step 1306) can entail comparing the first set of results with the second set of results. More particularly, the fourth step (step 1306) can entail comparing results of the first set of virtual measurements for one or more configurations with results of the second set of virtual measurements for the one or more configurations. For instance, the first and second sets of virtual measurements can be associated with measurements configured to evaluate the same set of biological attributes. In particular, the first set of virtual measurements can include a first virtual measurement, and the second set of virtual measurements can include a second virtual measurement. The first virtual measurement can be associated with a first measurement configured to evaluate a first biological attribute of a biological system absent the therapy. The second virtual measurement can be associated with a second measurement configured to evaluate the first biological attribute based on the therapy. In the present example, a result of the first virtual measurement for a configuration can be compared with a result of the second virtual measurement for the configuration. More particularly, comparing the result of the first virtual measurement for the configuration with the result of the second virtual measurement for the configuration can entail determining whether the result of the first virtual measurement differs from the result of the second virtual measurement. For certain applications, the first biological attribute can be identified as a biomarker that is predictive of biological activity of the therapy based on determining that the result of the first virtual measurement differs from the result of the second virtual measurement. In particular, the first biological attribute can be identified as exhibiting a change based on the therapy and can be evaluated for the biological system during the course of the therapy to assess biological activity of the therapy. Typically, results of the first virtual measurement for multiple configurations can be compared with results of the second virtual measurement for the multiple configurations to identify such biomarker. Use of multiple configurations can be desirable to allow verifying identification of such biomarker across the multiple configurations. For certain applications, comparing the results of the first virtual measurement for the multiple configurations with the results of the second virtual measurement for the multiple configurations can entail determining whether a result of the first virtual measurement for each configuration differs from a result of the second virtual measurement for the configuration.

In the present embodiment of the invention, identification of a biomarker can be verified using various methods. For certain applications, identification of a biomarker can be verified based on, for example, experimental or clinical data, scientific literature, results of a computer model, or a combination thereof. For instance, one or more additional virtual therapies can be defined to simulate different variations of the therapy (e.g., different dosages, treatment intervals, or treatment times), and the one or more additional virtual therapies can be processed as, for example, shown in FIG. 13 to verify identification of a biomarker with respect to the one or more additional virtual therapies. Alternatively, or in conjunction, one or more additional configurations can be defined, and identification of a biomarker can be verified by evaluating the behavior of the one or more additional configurations in a manner as described above.

### Using Identified Biomarkers

Once a biomarker has been identified, it can be used for various applications. For instance, a biomarker can be used to develop, test, and implement a therapy to treat a disease. For certain applications, a biomarker of a therapy for human patients can be used to perform a clinical trial of the therapy. For instance, a biological attribute can be identified as a biomarker that is predictive of a particular effect of the therapy, and a group of human patients can be selected for the clinical trial based on measurement of the biological attribute for the group of human patients. In particular, the biological attribute can be evaluated for a particular human patient absent the therapy to predict the degree of effectiveness of the therapy for the particular patient. The particular human patient can be selected for inclusion in the clinical trial based on whether measurement of the biological attribute indicates a sufficient degree of effectiveness of the therapy for the particular human patient.

As another example, a biological attribute can be identified as a biomarker that is predictive of effectiveness of a therapy, and measurement of the biological attribute can be performed for a group of human patients at one or more times during the course of a clinical trial. In particular, the biological attribute can be evaluated for a particular human patient during the course of the clinical trial to infer a surrogate end-point of the therapy for the particular human patient.

As a further example, a biological attribute can be identified as a biomarker that is predictive of biological activity of a therapy, and measurement of the biological attribute for a group of human patients can be performed at one or more times during the course of a clinical trial. In particular, the biological attribute can be evaluated for a particular human patient during the course of the clinical trial to assess biological activity of the therapy for the particular patient.

Each of the patent applications, patents, publications, and other published documents mentioned or referred to in this specification is herein incorporated by reference in its entirety, to the same extent as if each individual patent application, patent, publication, and other published document was specifically and individually indicated to be incorporated by reference.

While certain embodiments and examples have been described herein with reference to virtual therapies, it should be understood by one of ordinary skill in the art that embodiments of the invention are not limited to virtual therapies and, specifically, are not limited to the ability to identify biomarkers of a therapy. For instance, an embodiment of the invention can be used to identify biomarkers of various other types of stimuli. In particular, a virtual stimulus associated with a particular stimulus can be defined, and the virtual stimulus can be used in a manner as described herein to identify one or more biomarkers of the stimulus. A biological attribute that is identified as a biomarker of the stimulus can be evaluated to infer or predict a particular effect of the stimulus.

Also, an embodiment of the invention can be used to identify biomarkers of normal or disease conditions of a biological system. A biomarker of a normal condition (e.g., a healthy phenotype) typically refers to a biological attribute that can be associated with the normal condition. More particularly, a biomarker of a normal condition can refer to a biological attribute that can be evaluated to infer or predict a particular characteristic of the normal condition, such as a clinical sign or diagnostic criteria of the normal condition. In a similar manner, a biomarker of a disease condition (e.g., a disease phenotype) typically refers to a biological attribute that can be associated with the disease condition. More particularly, a biomarker of a disease condition can refer to a biological attribute that can be evaluated to infer or predict a particular characteristic of the disease condition, such as a clinical sign or diagnostic criteria of the disease condition. Biomarkers of normal or disease conditions can be used to diagnose diseases, to monitor disease progression, and to guide decision-making relating to treatment of diseases.

For such an embodiment, various configurations of a computer model can be defined to represent a normal condition, a disease condition, or both, of a biological system. The computer model can be executed to produce a set of results of a set of virtual measurements. In particular, the set of virtual measurements can be applied to one or more configurations to produce the set of results. Once produced, the set of results can be analyzed to identify one or more biomarkers. For instance, the set of virtual measurements can include a first virtual measurement and a second virtual measurement. The first virtual measurement can be associated with a first measurement for the biological system, and the second virtual measurement can be associated with a second measurement for the biological system. Here, the first measurement can be configured to evaluate a first biological attribute, and the second measurement can be configured to evaluate a second biological attribute that is indicative of the normal or disease condition. Results of the first virtual measurement for one or more configurations can be compared with results of the second virtual measurement for the one or more configurations to identify the first biological attribute as a biomarker that is predictive of the normal or disease condition. As another example, the set of virtual measurements can be applied to a first configuration and a second configuration of the computer model. Here, the first configuration can be defined to represent the normal condition, and the second configuration can be defined to represent the disease condition. Results of the set of virtual measurements for the first configuration can be compared with results of the set of virtual measurements for the second configuration to identify a biomarker that is predictive of the normal or disease condition.

An embodiment of the present invention relates to a computer storage product including a computer-readable medium having computer code thereon for performing various computer-implemented operations. As used herein, the term “computer-readable medium” can include any medium which is capable of storing or encoding a sequence of code or instructions for performing the processing described herein. The media and code may be those specially designed and constructed for the purposes of the present invention, or they may be of the kind well known and available to those having skill in the computer software arts. Examples of computer-readable media include, but are not limited to: magnetic media such as hard disks, floppy disks, and magnetic tape; optical media such as CD-ROMs and holographic devices; magneto-optical media such as floptical disks; carrier waves signals; and hardware devices that are specially configured to store and execute program code, such as application-specific integrated circuits (“ASICs”), programmable logic devices (“PLDs”), read only memories (“ROMs”), random access memories (“RAMs”), erasable programmable read only memories (“EPROMs”), and electrically erasable programmable read only memories (“EEPROMs”). Examples of computer code include machine code, such as produced by a compiler, and files containing higher level code that are executed by a computer using an interpreter. For example, an embodiment of the invention may be implemented using Java, C++, or other object-oriented programming language and development tools.

Moreover, an embodiment of the invention may be downloaded as a computer program product, where the program may be transferred from a remote computer (e.g., a server) to a requesting computer (e.g., a client) by way of data signals embodied in a carrier wave or other propagation medium via a communication link (e.g., a modem or network connection). Accordingly, as used herein, a carrier wave can be regarded as comprising a computer-readable medium.

Another embodiment of the invention may be implemented in hardwired circuitry in place of, or in combination with, machine-executable software instructions.

While the present invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, process step or steps, to the objective, spirit and scope of the present invention. All such modifications are intended to be within the scope of the claims appended hereto. In particular, while the methods disclosed herein have been described with reference to particular steps performed in a particular order, it will be understood that these steps may be combined, sub-divided, or re-ordered to form an equivalent method without departing from the teachings of the present invention. Accordingly, unless specifically indicated herein, the order and grouping of the steps is not a limitation of the present invention.

What is claimed is:

1. A computer-executable software code, comprising:
code to define a plurality of configurations associated with a computer model of a biological system, each configuration of the plurality of configurations being associated with a different representation of the biological system;
code to apply a virtual measurement to the plurality of configurations to produce a result of the virtual measurement for each configuration of the plurality of configurations, the virtual measurement being associated with a measurement for the biological system absent a therapy;
code to apply a virtual therapy to the plurality of configurations to produce a result of the virtual therapy for each configuration of the plurality of configurations, the virtual therapy being associated with the therapy; and
code to display the results of the virtual measurement for the plurality of configurations and the results of the virtual therapy for the plurality of configurations.

2. The computer-executable software code of claim 1, further comprising:
code to identify correlation between the results of the virtual measurement and the results of the virtual therapy.

3. The computer-executable software code of claim 2, wherein the code to identify correlation between the results of the virtual measurement and the results of the virtual therapy includes:
code to determine a correlation coefficient associated with the results of the virtual measurement and the results of the virtual therapy.

4. A computer-executable software code, comprising:
code to define a plurality of configurations associated with a computer model of a biological system, each configuration of the plurality of configurations representing a different combination of genetic and environmental factors for the biological system;
code to apply a virtual therapy to the plurality of configurations to produce, for each configuration of the plurality of configurations, a result of a first virtual measurement and a result of a second virtual measurement, the virtual therapy being associated with a therapy for the biological system, the first virtual measurement being associated with a first measurement for the biological system, the second virtual measurement being associated with a second measurement for the biological system, the second measurement being configured to evaluate effectiveness of the therapy; and
code to compare the results of the first virtual measurement for the plurality of configurations with the results of the second virtual measurement for the plurality of configurations.

5. The computer-executable software code of claim 4, wherein the code to compare the results of the first virtual measurement with the results of the second virtual measurement includes:
code to determine whether the results of the first virtual measurement are substantially correlated with the results of the second virtual measurement.

6. A computer-executable software code, comprising:
code to define a plurality of configurations associated with a computer model of a biological system, each configuration of the plurality of configurations being associated with a different representation of the biological system;
code to apply a first virtual measurement to the plurality of configurations to produce a result of the first virtual measurement for each configuration of the plurality of configurations, the first virtual measurement being associated with a first measurement for the biological system;
code to apply a second virtual measurement to the plurality of configurations to produce a result of the second virtual measurement for each configuration of the plurality of configurations, the second virtual measurement being associated with a second measurement for the biological system; and
code to display the results of the first virtual measurement for the plurality of configurations and the results of the second virtual measurement for the plurality of configurations.

7. The computer-executable software code of claim 6, further comprising:
code to compare the results of the first virtual measurement with the results of the second virtual measurement.

8. A computer-executable software code, comprising:
code to define a computer model of a biological system;
code to define a plurality of virtual measurements associated with the computer model, each virtual measurement of the plurality of virtual measurements being associated with a different measurement for the biological system, the plurality of virtual measurements including a first plurality of virtual measurements and a second plurality of virtual measurements;
code to define a virtual therapy, the virtual therapy being associated with a therapy for the biological system;
code to execute the computer model absent the virtual therapy to produce a first plurality of results of the first plurality of virtual measurements;
code to execute the computer model based on the virtual therapy to produce a second plurality of results of the second plurality of virtual measurements; and
code to display the first plurality of results of the first plurality of virtual measurements and the second plurality of results of the second plurality of virtual measurements.

9. The computer-executable software code of claim 8, wherein the computer model represents a plurality of biological processes of the biological system using a plurality of mathematical relations, and the code to define the virtual therapy includes:
code to define the virtual therapy as a parametric change in at least one mathematical relation of the plurality of mathematical relations.

10. The computer-executable software code of claim 8, further comprising:
code to compare the first plurality of results with the second plurality of results.

11. A method of identifying a biomarker of a therapy for a biological system, comprising:
executing a computer model of the biological system to produce a result of a first virtual measurement for each configuration of a plurality of configurations associated with the computer model, the first virtual measurement being associated with a first measurement for the biological system, each configuration of the plurality of configurations being associated with a different representation of the biological system;
executing the computer model based on a virtual therapy to produce a result of a second virtual measurement for each configuration of the plurality of configurations, the virtual therapy being associated with the therapy, the second virtual measurement being associated with a second measurement for the biological system, the second measurement being configured to evaluate an effect of the therapy; and
comparing the results of the first virtual measurement for the plurality of configurations with the results of the second virtual measurement for the plurality of configurations.

12. The method of claim 11, wherein executing the computer model to produce the results of the first virtual measurement for the plurality of configurations includes:
executing the computer model based on a virtual stimulus to produce the results of the first virtual measurement for the plurality of configurations, the virtual stimulus being associated with a stimulus for the biological system.

13. The method of claim 11, wherein the computer model represents a plurality of biological processes of the biological system, the method further comprising:
identifying a biological process of the plurality of biological processes that modulates biological response to the therapy; and
defining each configuration of the plurality of configurations as being associated with a different modification of the biological process.

14. The method of claim 11, wherein comparing the results of the first virtual measurement with the results of the second virtual measurement includes:
determining a correlation coefficient associated with the results of the first virtual measurement and the results of the second virtual measurement.

15. The method of claim 14, wherein the first measurement is configured to evaluate a biological attribute of the biological system, the method further comprising:
identifying the biological attribute as a biomarker of the therapy based on the correlation coefficient.

16. A method of identifying a biomarker of a therapy for a biological system, comprising:
defining a plurality of configurations of a computer model of the biological system, each configuration of the plurality of configurations being associated with a different representation of the biological system;
applying a virtual therapy to the plurality of configurations to produce, for each configuration of the plurality of configurations, a result of a first virtual measurement and a result of a second virtual measurement, the virtual therapy being associated with the therapy, the first virtual measurement being associated with a first measurement for the biological system, the second virtual measurement being associated with a second measurement for the biological system, the second measurement being configured to evaluate effectiveness of the therapy; and
comparing the results of the first virtual measurement for the plurality of configurations with the results of the second virtual measurement for the plurality of configurations.

17. The method of claim 16, further comprising:
validating the plurality of configurations with respect to a given phenotype of the biological system.

18. The method of claim 16, wherein comparing the results of the first virtual measurement with the results of the second virtual measurement includes:
determining that the results of the first virtual measurement are substantially correlated with the results of the second virtual measurement.

19. The method of claim 18, wherein the first measurement is configured to evaluate a biological attribute of the biological system, the method further comprising:
identifying the biological attribute as a biomarker that is predictive of effectiveness of the therapy for the biological system.

20. A method of identifying a biomarker for a biological system, comprising:
executing a computer model of the biological system to produce, for each configuration of a plurality of configurations associated with the computer model, a result of a first virtual measurement and a result of a second virtual measurement, each configuration of the plurality of configurations being associated with a different representation of the biological system, the first virtual measurement being associated with a first measurement for the biological system, the second virtual measurement being associated with a second measurement for the biological system; and
comparing the results of the first virtual measurement for the plurality of configurations with the results of the second virtual measurement for the plurality of configurations.

21. The method of claim 20, wherein comparing the results of the first virtual measurement with the results of the second virtual measurement includes:
determining a correlation coefficient associated with the results of the first virtual measurement and the results of the second virtual measurement.

22. The method of claim 21, wherein the first measurement is configured to evaluate a biological attribute of the biological system, the method further comprising:
identifying the biological attribute as a biomarker based on the correlation coefficient.

23. The method of claim 22, wherein the biomarker is predictive of one of a normal condition and a disease condition of the biological system.

24. A method of performing a clinical trial of a therapy, comprising:
applying a virtual measurement to a plurality of virtual patients associated with a computer model to produce a result of the virtual measurement for each virtual patient of the plurality of virtual patients, the virtual measurement being associated with measurement of a biological attribute absent the therapy, each virtual patient of the plurality of virtual patients being associated with a different human patient;
applying a virtual therapy to the plurality of virtual patients to produce a result of the virtual therapy for each virtual patient of the plurality of virtual patients, the virtual therapy being associated with the therapy;
comparing the results of the virtual measurement for the plurality of virtual patients with the results of the virtual therapy for the plurality of virtual patients to identify the biological attribute as predictive of effectiveness of the therapy; and
selecting a group of human patients for the clinical trial based on measurement of the biological attribute for the group of human patients.

25. The method of claim 24, wherein the computer model represents a plurality of biological processes, the method further comprising:
identifying a biological process of the plurality of biological processes that modulates biological response to the therapy; and
defining each virtual patient of the plurality of virtual patients as being associated with a different modification of the biological process.

26. The method of claim 24, wherein comparing the results of the virtual measurement with the results of the virtual therapy includes:
determining a correlation coefficient associated with the results of the virtual measurement and the results of the virtual therapy.

27. A method of performing a clinical trial of a therapy, comprising:
receiving an identification of a biological attribute as being predictive of effectiveness of the therapy, the identification of the biological attribute being based on a computer-based simulation of a plurality of virtual patients absent a virtual therapy and a computer-based simulation of the plurality of virtual patients based on the virtual therapy, the virtual therapy being associated with the therapy, each virtual patient of the plurality of virtual patients being associated with a different human patient; and
selecting a group of human patients for the clinical trial based on measurement of the biological attribute for the group of human patients.
